(19) United States
(12) Patent Application Publication
(10) Pub. No.: US 2005/0106564 A1
(43) Pub. Date: May 19, 2005

(54) One-step, no-wash multiplex bead-based flow cytometric assay

(76) Inventor: Mark K. Hechinger, Inglewood, CA (US)

Correspondence Address: Colin P. Abrahams, 5850 Canoga Avenue, Suite 400, Woodland Hills, CA, 91367, US

(21) Appl. No.: 10/949,938

(22) Filed: Sep. 24, 2004

Related U.S. Application Data
(63) Continuation-in-part of application No. 09/873,866, filed on Jun. 4, 2001.
(60) Provisional application No. 60/209,437, filed on Jun. 2, 2000.

(51) Int. Cl. C12Q 1/70

(52) U.S. Cl. 435/5

(57) ABSTRACT

A no-wash multiplex bead system is disclosed in which all basic reaction materials can be placed into single container via manual application, robotic transfer, or any other batch-type testing procedure and allowed to incubate followed by analysis on a flow cytometer.

### CROSS REFERENCE TO RELATED APPLICATIONS

This application is a continuation in part of U.S. patent application Ser. No. 09/873,866 filed Jun. 4, 2001, which claims the benefit of U.S. Provisional Patent Application No. 60/209,437 filed Jun. 2, 2000, all of which are incorporated herein by reference in its entirety.

### FIELD AND BACKGROUND OF THE INVENTION

Multiplex bead-based assays are designed to be utilized on flow cytometry instrumentation and to provide rapid, efficient means of detecting immunological interactions. Nevertheless, most of these assays require multiple wash steps between the addition of sample and the fluorescent indicator (also called the conjugate). These multiple wash steps increase the technical complexity of the procedure and also increase the risk of procedure error.

Immunological assays have developed, over the past twenty years, into high volume, low cost evaluations to determine immune competency in patients. Whether quantitating the amount of antibody in a sample, or detecting the presence of viral particles, these serological assays are now commonplace in many clinical laboratories. Unfortunately, however, these tests are often encumbered with long incubations and extensive technical preparation time and lengthy washing processes, thus wasting countless hours and costing the laboratory in personnel time, as well as in materials. Therefore, the first completely “No-wash” assay has been developed to not only save time and money, but also to improve upon the overall technical accuracy when compared with traditional assay methods.

Enzyme-linked Immunosorbant assays, or Elisa's, have been the common format for many serum-based tests. Whether for detecting antigens or antibodies in patient samples, this technology bases its principles on the interactions between a bound antigen on the surface of plastic microtiter plate wells, detector antibodies labeled with a chemically inert substrates (for example, horseradish peroxidase), and developer reagents (i.e. OPD) which cause the antibody labels to change color when present in the form of antigen-antibody complexes. These assays can become very cumbersome. Not only is there a requirement to wash between incubations, but most such assays are time dependent and will over incubate (e.g. increase intensity of color) if left too long, and, as a result thereof, often be too difficult, if not impossible, to analyze. Therefore, they are extremely time dependent assays and require careful monitoring to ensure that the reactions do not produce falsely positive results.

Another disadvantage to these assays is the instability of their reagents and the inconvenience of mixing buffers and reconstituting enzyme solutions prior to incubation. Errors may occur if any reagent is improperly mixed, added incorrectly, or allowed to contact the reaction surface for any length of time not stipulated by the manufacturer. Stopping reagents are often included in the assay kits. Furthermore, these assays are also light sensitive and will “bleach” when exposed to long periods of light.

### SUMMARY OF THE INVENTION

Continuing with the older technologies, such as Elisa, appears not to be the most efficient platform. In this time of Diagnosis Related Groups (DRG's), efficiency and “clinical necessity” are the key words in laboratory economics. The present invention, in one aspect, describes a method by which laboratory technical personnel may simply pre-dilute a patient's serum, add a specific amount to the reaction material, add conjugate and wait for a pre-determined length of time and “read” (i.e. analyze) the assay. All of this can be achieved in one step. This saves time and money and eliminates some of sources of error which common plague other assays.

In one aspect of the present invention, the user does not need to wash at all. Using the properties of the flow cytometer, the user may simultaneously add beads, sample, and indicator fluorochrome into the same reaction vessel with no intermediate washing steps. Such a process can improve the light scatter properties, otherwise known as forward scatter (size) and side scatter (complexity), reduce the inherent clumping of the bead populations, while still greatly reduce the non-specific background fluorescence normally seen in multiplex bead applications with wash steps. Furthermore, the method may allow for modification of the procedure to accommodate microtiter plate applications. This fits most modern analysis platforms using flow cytometry.

The method of the invention can be applied to any bead-based application and is not dependent upon the number of bead sizes per reaction vial. The two examples in the present application, provided below, use a single bead and multiple beads in one reaction vessel for the purpose of detecting antibodies to H. Pylori antigen and ENA antigens, respectively. However, this process can be used in a number of research and clinical applications and have a variety of bead sizes, numbers, and indicator fluorochromes used.

The invention utilizes, in one aspect, a multiplex micro-bead technology and is developed to (1) utilize small volumes of patient sample and reagents, (2) be compatible to microtiter tray analysis platforms for high throughput, and (3) be as rapid an assay as possible without the need for additional technical supervision. Unlike other bead-based systems, the invention maintains a true one-step, no-wash procedure in which all reagents may be added to a single vessel (such as, for example, a microtiter plate, test tube, etc.) simultaneously, incubated, and analyzed on the flow cytometer.

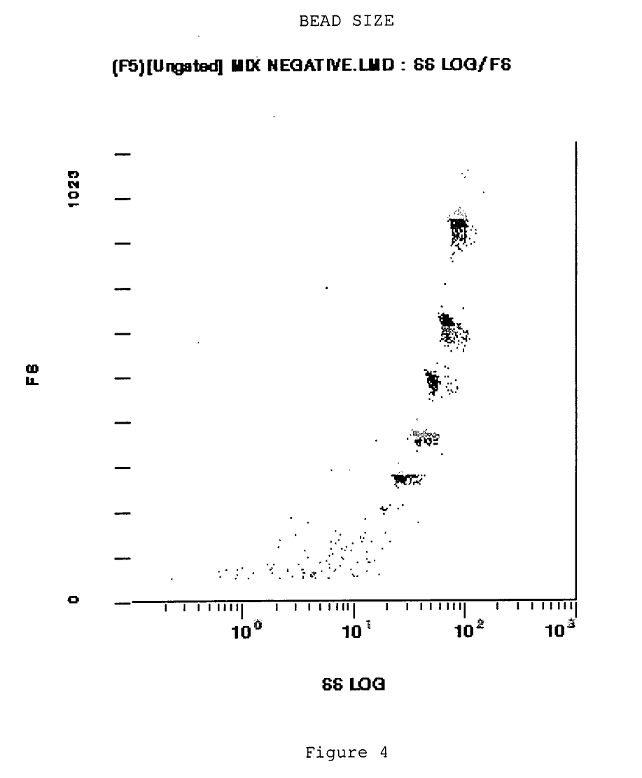

The size characteristics and/or a combination of size and fluorescent properties of the beads distinguish them from each other. On flow cytometers, this is accomplished by utilizing Forward Scatter properties (size) together with Side Scatter (SSC) (see FIG. 4), which is a measurement of the refractive properties of particles passing in front of a light source, which is generally a laser. These signals are combined to form scatter plots by the host computer, which may be utilized in the identification of bead population(s). Furthermore, inherent or secondary bead fluorescence may be used as an additional means of “gating” selected populations. In combination with each other, multiple properties of size, side scatter, and fluorescence, create unlimited possibilities of analytes detected per sample.

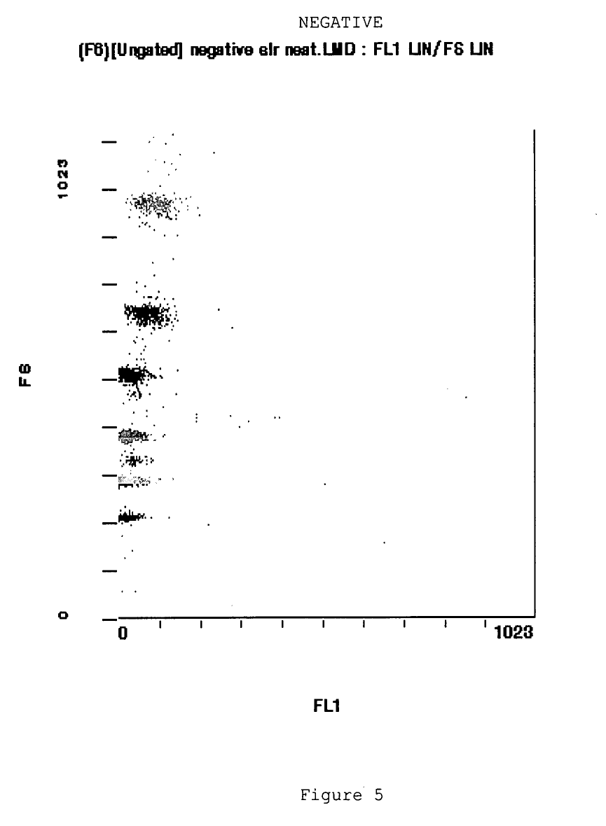

Specific volumes of beads solution, pre-diluted patient serum, and conjugate can be simultaneously mixed together in a single reaction vessel, incubated and analyzed in a very short period of time. The present invention has been developed from experienced flow cytometry users and has been formulated in an effort to eliminate most of, or a significant amount of, the bead clumping and/or background fluorescence known to be associated with any multiplex assay, let alone a “no wash” system. Proportional bead coating and antigen/bead ratios have been determined for many assays allowing the beads to be distinct populations when viewed on the flow cytometers. Similarly, the “normal” or negative controls used with these assay systems are clearly distinguished from “positive” samples (See FIGS. 5 & 6). Results are reported either as a qualitative result (positive or negative), using specific mean channel cut-off or as semi-quantitative values by dividing the mean channel fluorescence of the positive sample by the mean channel of the negative control. This creates the potential for monitoring serum titers of the specific analyte. In most assays, an index of 1.4-1.5 is considered positive (Table 1). Quantitative results may also be incorporated by utilizing known multiple positive control standards, which may form concentration curves when plotted on a graph of result versus concentration value.

In conclusion, the invention has, in one aspect, found specific ways in which to make multiplex bead technology the preferred or optimal scenario for any immunological testing process. Having “no wash” technology equated into the standard laboratories operation, reduces wasted personnel time, increases testing throughput, potentially eliminates the chances of improper reagent application, is flexible enough to be utilized on the newer microtiter plate analysis platforms, which opens the potential use of auto- or robotic reagent dispensers and is also flexible enough to combine any antigen/antigen, bead, or conjugate combination.

### DETAILED DESCRIPTION OF THE INVENTION

In accordance with one preferred embodiment of the present invention, antigen coated latex particles of various sizes and combinations therein, fluorescenated indicator antibodies, sample or body fluid, and flow cytometers are combined to provide a device that requires the addition of all the reagents above into one reaction vessel. After waiting for an incubation time period to pass, an analysis takes place on the flow cytometer without multiple steps, incubations, or intermediate washings to remove excess reagents.

One aspect of this invention is to develop antibody/antigen detection kits that will simplify the process of analyzing samples for the presence of these analytes. Latex beads are coated with antigens or antibodies to their exterior. There may be only one bead, or there may be multiple sized beads. Each bead may be coated with one or multiple antigens/antibodies, or the latex may or may not be impregnated with specific dyes. Nevertheless, the process of this invention, in one aspect, is unique from other bead-based assays in that it allows the coated beads, sample or reaction material, and indicator conjugate to be simultaneously added to one reaction vessel, whether a microtiter tray or test tube, thereafter incubated for a given amount of time, and then analyzed on a flow cytometer without inter-step washings as with ELISA technology.

The uncoated beads are washed prior to coating with the specific antigen/antibody to its surface. The supernatant, containing excess antigen, liquid and suspended beads, is decanted from the packed beads on the bottom of the centrifuge tube. The remaining material is gently resuspended, after which a specific amount of antigen (defined as any entity that is the target of an immunological reaction) is applied to the concentrate on the bottom of the centrifuge tube. This antigen/bead slurry is vortexed and resuspended in a given volume of coating buffer, incubated and then centrifuged and decanted. The second resuspension may include coating the beads with a protein solution to eliminate excess background binding of non-specific antibodies or other proteins. If so, this is quickly added, vortexed with the beads, and centrifuged to remove the supernatant. The reaction beads are then resuspended in a buffer solution, which may, or may not, contain a protein additive or preservative. This is store refrigerated until ready for use. Multiple needs of the specific assay may be prepared in accordance with the procedure described above.

The beads maybe aliquoted into reaction vessels and have specific amounts of pre-diluted sample added to them. Shortly thereafter, an indicator conjugate is added to the mixture, incubated and analyzed on the flow cytometer. Bead/sample mixtures are aspirated by the flow cytometer, pass through a flow cell, and are analyzed and distinguished by the bead(s)scatter properties when presented in front of light source produced from laser(s) found within the flow cytometer itself. Bead populations are distinguished from each other by their size, light scattering, and fluorescent properties. Each bead has a unique scatter characteristic, which is identified by forward angle light scatter (size) together with side angle scatter (refractal) properties. These signals are converted into a digital signal, which are then graphically plotted on a two-dimensional histogram where each population may be delineated by drawing specific “gate(s)”, or windows. Information (for example, fluorescence) regarding events within these “gate(s)” is then transmitted to other individual plots, or histograms, to determine their properties (for example, positive or negative, bright or dim, etc.). Negative control samples are initially analyzed to determine inherent fluorescent properties, or background. This signal, or mean channel fluorescence, will be the denominator of the assay result itself. Positive samples will form reaction complexes with the beads and “shift” the position (mean channel fluorescence) of the population in relation to that of the negative control on the histogram.

Mean channel fluorescent measurements are defined as the relative position of the population of the beads found on a histogram. The scaling of these histograms may be different from vendor to vendor. However, most flow cytometers have the ability to scale linear histograms from 0 to 1023 channels. This number may be utilized as an indicator of the relative degree of “positivity” to that of a normal or “negative” sample, therefore allowing the potential for semi-quantitative results. For example, if the mean channel of the negative sample were at channel 50 and the positive was channel 250, the semi-quantitative index (result) of the positive sample would be 5.0 (i.e. 250/50). This has wide clinical implications and eventually would be useful when a true quantitative panel comprised of standardized control samples was implemented. Results to clinicians would help monitor disease status or the effectiveness of therapeutic regimens.

Therefore, this truly “no-wash” bead technology is also of considerable importance to other applications of multiplex technology. Other processes have had difficulty with bead clumping, increased noise signals created by the protein/conjugate interactions, reduced shelf life, improper antigen/antibody binding from competitive reactions found within assay process, and variable results after prolonged incubation.

Some of the benefits of the present invention can be described as follows:
(1) Absolutely no intermediate wash steps which saves personnel labor and the laboratory money.
(2) Decreases time needed to obtain a result and, therefore, the clinician, or person ordering the test, has results faster.
(3) There is less chance of technical error when all reagents are added simultaneously.
(4) Unlike ELISA technology, the “no-wash” bead product does not over-develop and change color to the point where the technical staff could not analyze the samples. It is generally very stable.
(5) Unlike most other bead assays, the “no-wash” system is light stable.
(6) The simplicity of the reagents allows for modifying the assay towards automated/robotic-type reagent dispensing systems in larger laboratories.
(7) Data from the flow cytometer may be transferred to any laboratory computer system, or laboratory information system (LIS), automatically.
(8) The configuration of the procedure allows for the reaction to occur within test tubes, microtiter plates, or any other sample container requiring multi-process containers.
(9) Small amounts of sample are required for the assay, thus making it convenient for the analysis of hard-to-acquire/find samples.
(10) The “no-wash” technology has developed ways to decrease, if not eliminate, bead clumping in the presence of multiple reagents. This creates a cleaner, more distinguished population.
(11) The “no-wash” invention may be utilized on any clinical flow cytometer.
(12) Mean channel fluorescence may be utilized to form semi-quantitative results using the mean channel of the sample divided by the mean channel of the negative or “normal” control. Quantitative results would be calculated in a similar fashion using known control standards and plotting the sample index against that of the known concentration.
(13) Any antigen may be attached to the bead(s). Each assay may contain one or more beads and have one or more indicator systems involved.
(14) Antigens selected to be attached to the bead(s) surface (s) may be antigens, antibodies, chemicals, microorganisms, cell components, and/or other substances capable of binding specifically to an appropriate ligand, including DNA and RNA for in situ hybridization, primer strands from PCR assays and rare event antigens from esoteric assays.
(15) A relatively unlimited range of combinations using bead sizes and fluorescenated indicator dyes may be used within the scope of the invention.
(16) Semi-quantitative and quantitative results may be generated from the flow cytometer.

The invention will now be described with reference to some illustrative but not limiting examples.

### EXAMPLE 1

No Wash All-in-One Immunoassay System

In accordance with one example of the present invention, antigen coated latex beads, sample, and fluorescenated indicator reagents are combined into a single reaction vessel and allowed to incubate without additional inter-step washings to remove excess analytes. Antigens, as used below, may be recombinant proteins, antibodies, viral proteins, chemicals, bacterial proteins, or any other entity possessing the ability of binding to a latex bead surface.

The following steps may be utilized.
(1) Select from various sizes of beads to be used in the assay.
(2) Obtain antigens to be coated onto each bead or a combination of antigens therein.
(3) Pre-washing, by centrifugation, a specific amount of bead material in a buffer solution.
(4) Decanting the supernatant from the bead “pellet”.
(5) Gently resuspending the bead concentrate.
(6) Applying the antigen directly to the bead concentrate.
(7) Gently vortexing the bead/antigen solution.
(8) Applying another volume of buffer solution.
(9) Allowing the bead/antigen solution to incubate.
(10) Removing excess antigen and buffer by centrifugation.
(11) Carefully decanting supernatant from bead concentrate.
(12) Gently vortexing the bead/antigen concentrate.
(13) Applying a specific amount of protein solution to the bead/antigen concentrate (optional depending on antigen utilized)
(14) Gently vortexing the bead/antigen solution.
(15) Removing excess antigen and protein solution by centrifugation.
(16) Decanting the supernatant from the bead/antigen “pellet”.
(17) Gently vortexing the bead/antigen solution.
(18) Resuspending the bead/antigen mixture to a final assay concentration using buffer.
(19) Taking a given amount of the assay mixture and placing into a reaction vessel.
(20) Adding a specific amount of the sample to be analyzed into the reaction vessel.
(21) In addition, adding the indicator reagent to the same reaction vessel.
(22) Gently vortexing and incubating the bead/antigen suspension, sample, and indicator solution.
(23) Analyzing on the flow cytometer.

### EXAMPLE 2

Anti-ENA, No-Wash Detection Assay

In accordance with the present invention, multiple purified antigens RnP/Sm, Sm, SS-A, SS-B, and Scl-70 are incubated with bead sizes of 4, 5, 10, 7, and 3 microns, respectively, and stabilized for an extended shelf life. Diluted patient serum is then placed with 200 uLs of the bead mixture along with 50 uLs of Goat Anti-human IgG f(ab′)² FITC conjugated antibody diluted 1:20 in 1% bovine albumin in a pH 7.4 carbonate buffer. This reaction is allowed to incubate for a minimum of 30 minutes and thereafter analyzed on the flow cytometer.

### EXAMPLE 3

Anti-dsDNA, No-Wash Detection Assay

In accordance with the present invention, 5 u latex beads are coated with purified dsDNA. Patient samples are pre-diluted in carbonate buffer pH 7.4. A 10 uL of sample is added to 200 uL of dsDNA bead suspension after which, 50 uL of Goat Anti-human IgG f(ab′)² FITC conjugated antibody diluted 1:20 in 1% bovine albumin in pH 7.4 carbonate buffer is added and incubated Flow cytometric analysis follows. Positivity is determined by the relative degree of fluorescence over and above that of the normal, or negative, control. An index may be devised by dividing the mean channel fluorescence of the sample by the mean fluorescence of the normal control.

### EXAMPLE 4

A No-Wash Assay for the Detection of Antibodies to H. Pylori Antigen

In accordance with the present invention, H. Pylori antigen is coated onto a specific sized bead. Serum from a patient(s) is prediluted with buffer and applied to a specific amount of coated bead suspension. In addition, a multiple conjugate cocktail of anti-human IgG, IgA and IgM, each with unique fluorochromes, is added to the bead/serum mixture. Vortexing and incubation then follows. Analysis of the reaction mixture on the flow cytometer yields three separate results for antibodies to H. Pylori of classes IgG, IgA and IgM, simultaneously.

Instrument Set-up and Analysis

Flow cytometry utilizes size parameters to distinguish between individual cell populations. After discriminating specific areas of importance, flow cytometers rely on fluorescent properties produced by the addition of specific reagents (antibody or stain), which may produce one or multiple emission wavelengths.

Three simple steps are generally required for any of the bead assays. First, perform all necessary quality control daily procedures to comply with manufacturer specifications including regularly scheduled maintenance. Next, proceed to adjust size settings based on the instructions which may be provided. Finally, adjust to the assay settings and begin running samples. It is an easy and convenient way to evaluate all immunoassay procedures.

Beads Quality Assurance Kit

Bead technology offers a convenient way of insuring that a clinical flow cytometer apparatus is performing at the level necessary to utilize the bead technology. Forward scatter and fluorescence standards are available in custom configurations depending on your assay.

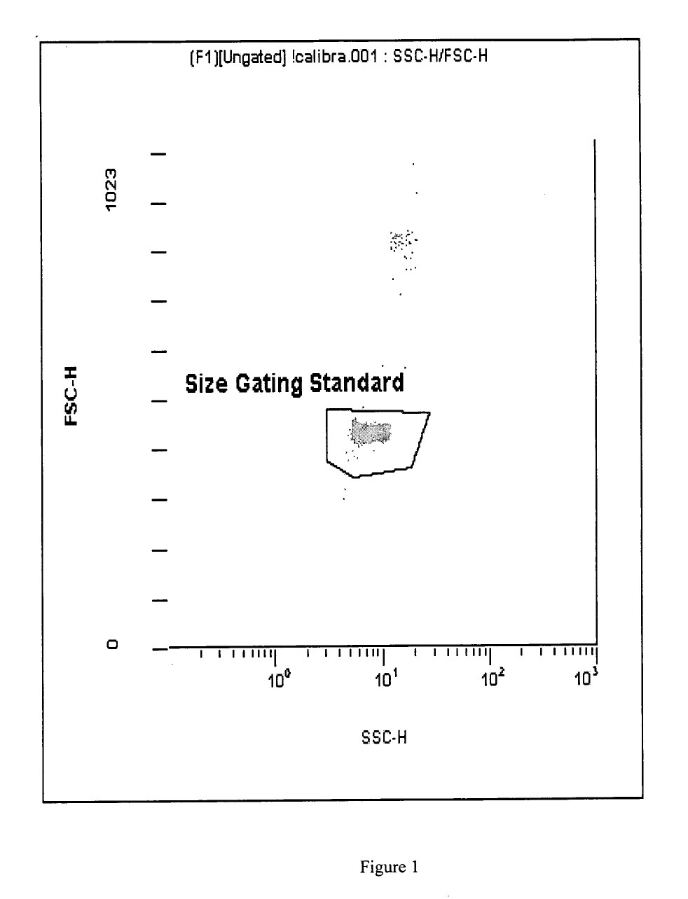

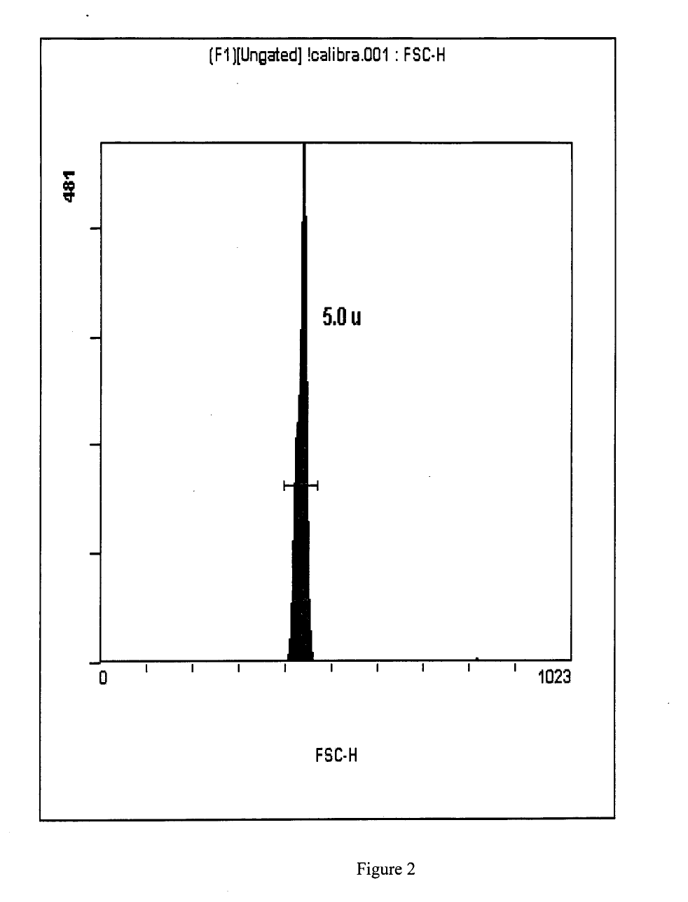

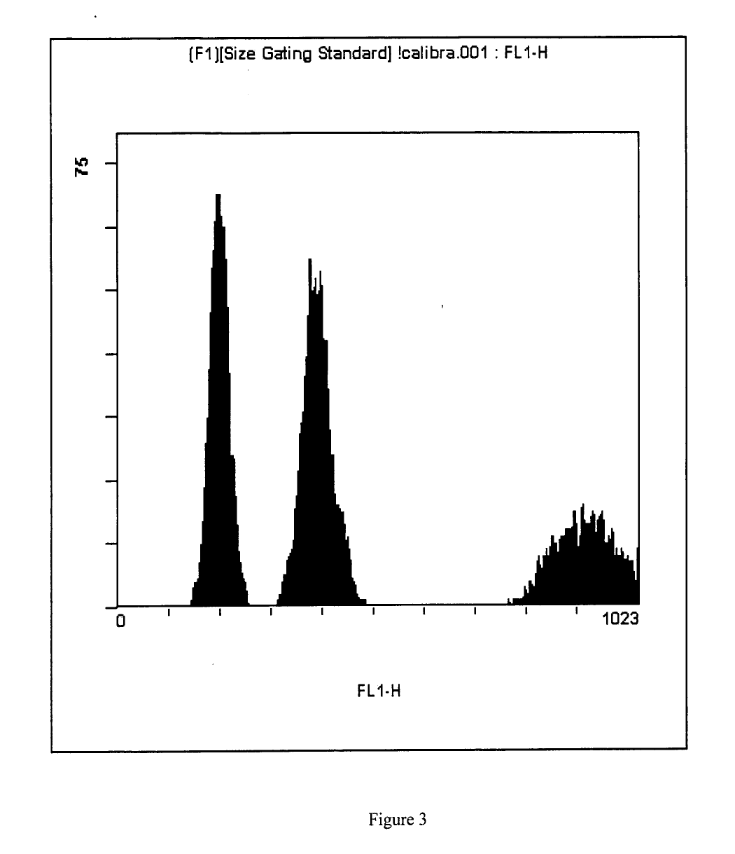

The bead fluorescent control of the present invention monitors a preferably centrally sized, three level fluorescent particle, which may be used to adjust both the forward scatter (size) and FL1 PMT settings. See FIGS. 1, 2 and 3.

One preferred procedure is as follows:
(1) The template provided is copied into the flow cytometer instrument protocol folder.
(2) Conveniently, this protocol folder may be named “Calibration Beads”.
(3) The flow cytometer is connected.

(4) The Instrument settings window opened and preferably adjusted as follows:

| Parameter | Detector | Voltage | AmpGain | Mode |
|---|---|---|---|---|
| P1 | FSC | E00 | 7.85 | LIN |
| P2 | SSC | 220 | 1.00 | LOG |
| P3 | FL1 | 693 | 6.30 | LIN |

These adjustments are approximations only and may need to be modified depending on the fluidic and laser power. Each manufacturer is different and may require different instrument settings. The above settings have been taken from a BD Biosciences FACS Calibur.
(5) Take 0.250 mL of calibration bead material and place it into a 12×75 mm test tube.
(6) Label the sample as necessary in the ID field.
(7) Place the test tube containing the Calibration material on the analysis station of the flow cytometer.
(8) Under “Set-up” mode adjust the scatter gate to encompass all of the bead population.
(9) Adjust the FLl PMT to approximate a mean channel of 200 for the first of the three fluorescent peaks.
(10) Save these settings.
(11) These parameters will remain throughout the lot of calibration beads.
(12) Record the mean channels in daily quality control logbook.
   Flow Cytometer Beads Anti-ENA Antibody Detection Kit

This bead line of products utilizes a method, in accordance with the invention, of stabilizing multiple beads in a single suspension and therefore optimizing their shelf life. Of these products, the Anti-ENA Detection Kit is the first in a series of convenient no-wash assays that can be utilized on any clinical flow cytometer. No additional equipment needed.

Antibodies against RnP/Sm, Sm, SS-A, SS-B, and Scl-70 can be simultaneously detected in one test tube using bead-sizing techniques. More specific kits are available for RnP/Sm and Sm only, SS-A and SS-B only, Scl-70 only and dsDNA only.

Similarly to that of the calibration material, the Flow Cytometer Bead Assay for Anti-ENA Antibodies protocol as described herein. Once calibration has been completed, the operator is able to format the flow cytometer to use any of the multiple analyte bead products. The steps below are then followed to analyze results.

Procedure:

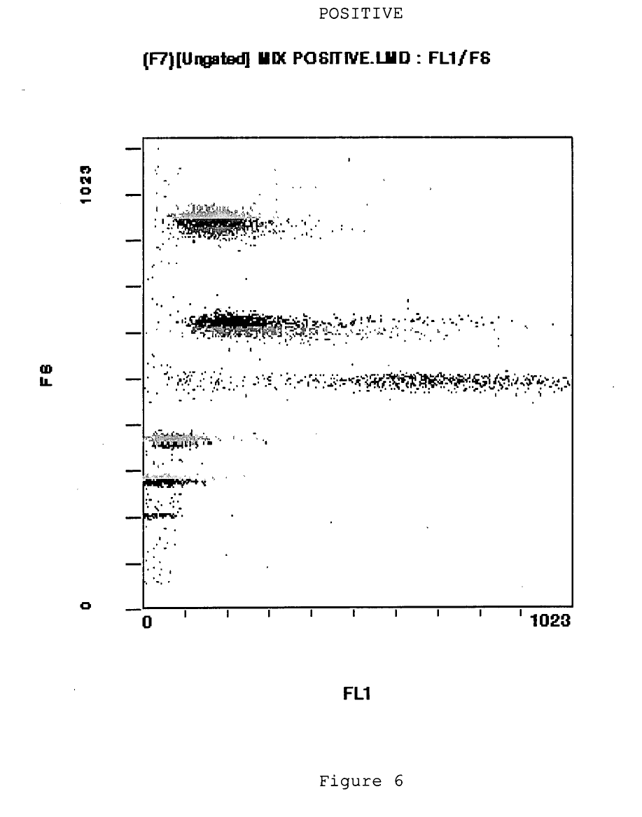

(1) Copy protocol template. See FIGS. 4, 5 and 6. These FIGS. 4, 5, and 6 represent data collected on a Beckman Coulter XL instrument. Analysis is performed using forward scatter versus fluorescence (linear) on last two cytograms.

(2) The instrument settings “SpheroFlow ENA Assay” are copied to the Instrument Settings file.

(3) The preliminary settings should be as follows:

| Parameter | Detector | Voltage | AmpGain | Mode |
|---|---|---|---|---|
| P1 | FSC | E00 | 7.85 | LIN |
| P2 | SSC | 233 | 1.00 | LOG |
| P3 | FL1 | 605 | 2.85 | LIN |

Note: These values may vary from instrument to instrument depending on laser power and fluidics. However, the results may be normalized and compared to other facilities.

(4) Label two (2) test tubes for normal and positive controls as well as any patient sample names. These will be the assay tubes.

Note: Microtiter trays may be substituted for test tubes.

(5) Label an identical set of tubes for all the patients. These will be the dilution tubes.

(6) Add 1 mL of sample diluent to each of the patient dilution tubes.

(7) Add 10 uL of each patient sample to the corresponding dilution tube.

(8) Vortex all dilution tubes until properly mixed.

(9) Into each of the assay tubes, including the negative and positive controls, add 200 uLs of Anti-ENA bead solution.

(10) Into each of the assay tubes, add 10 uLs of negative control, positive control, and patient samples into their corresponding tubes.

NOTE: The negative and positive controls should not be diluted. These are prediluted to offer a “low” positive result. Further dilutions may well invalidate results.

(11) Place one drop (approx. 50 uL) of assay conjugate solution in each test tube.

(12) Gently vortex the bead/sample/conjugate Solution.

(13) Incubate for at least 30 minutes at room temperature, in the dark.

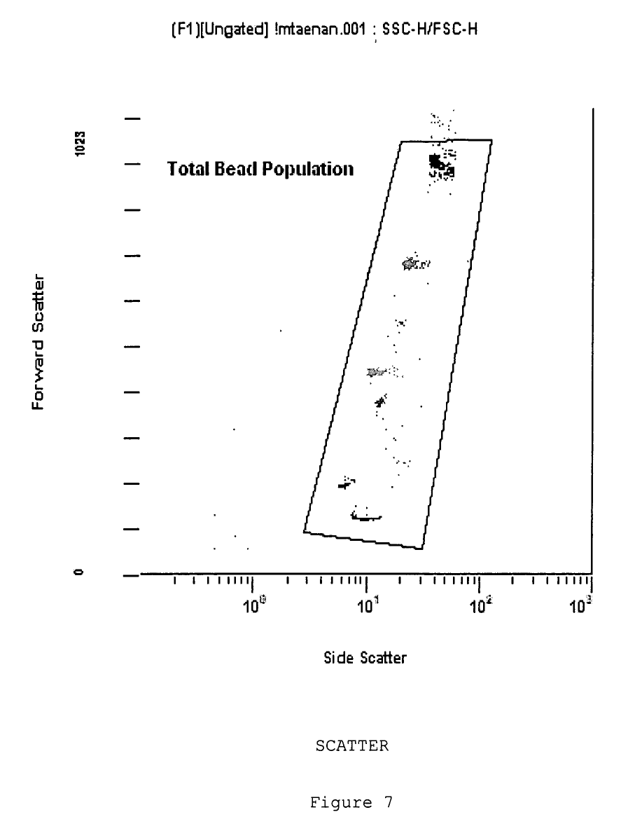

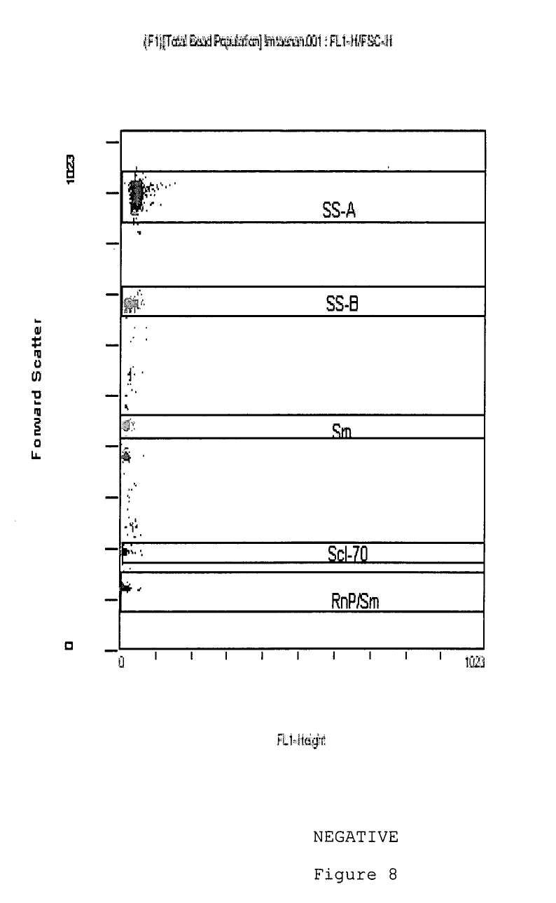

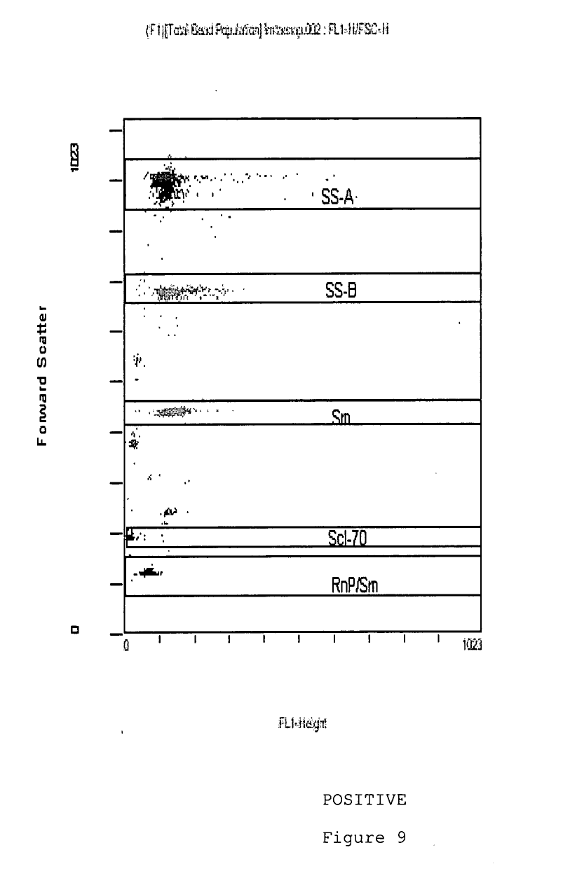

(14) Utilizing the Negative Control, establish that the smaller sized bead (RnP/Sm) has a background mean channel fluorescence between 30 and 40. This should remain consistent unless other problems exist with the laser and fluidics. See FIGS. 7, 8 and 9.

(15) A positive result is calculated by dividing the mean channel of the negative control, individual assay, by the corresponding positive control result. For example, if the patient sample has a RnP/Sm mean channel of 32 and the negative for RnP/Sm is 16, the index will be 2.0.

Note: The positive control samples have been selected as cut-off low controls. Some background debris may be present. Patient samples will be “cleaner” than expected.

Flow Cytometer Beads Anti-dsDNA Antibody Detection Kit

(1) Repeat steps 1-13 in Anti-ENA protocol.

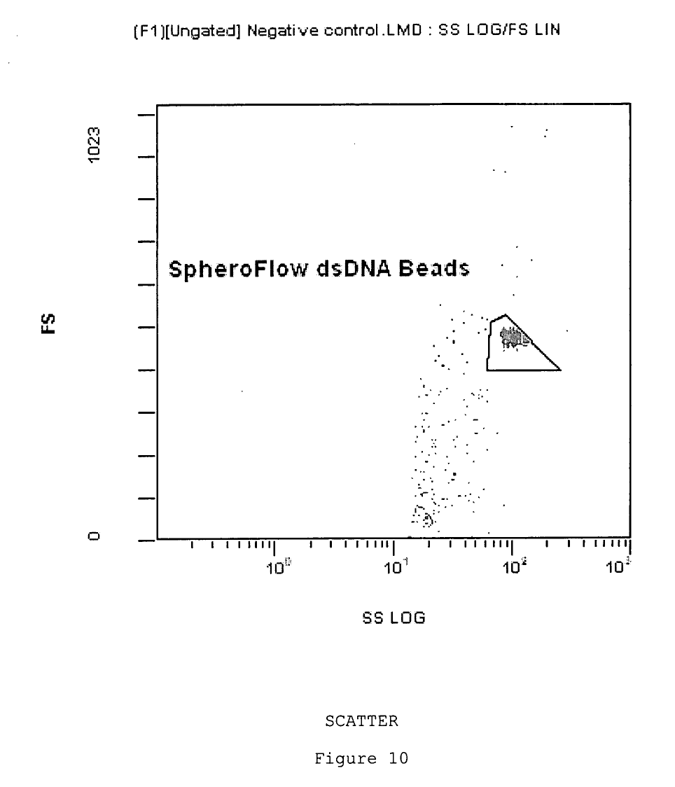

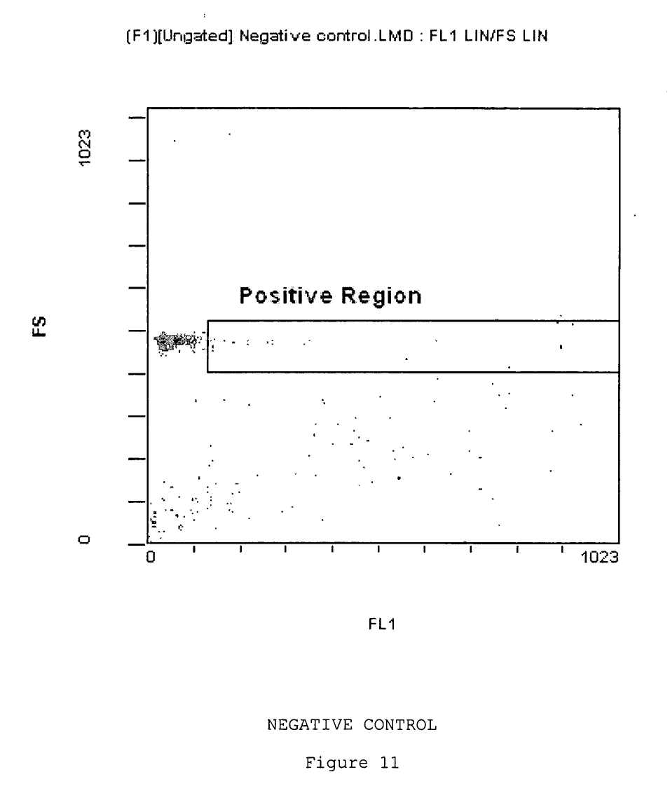

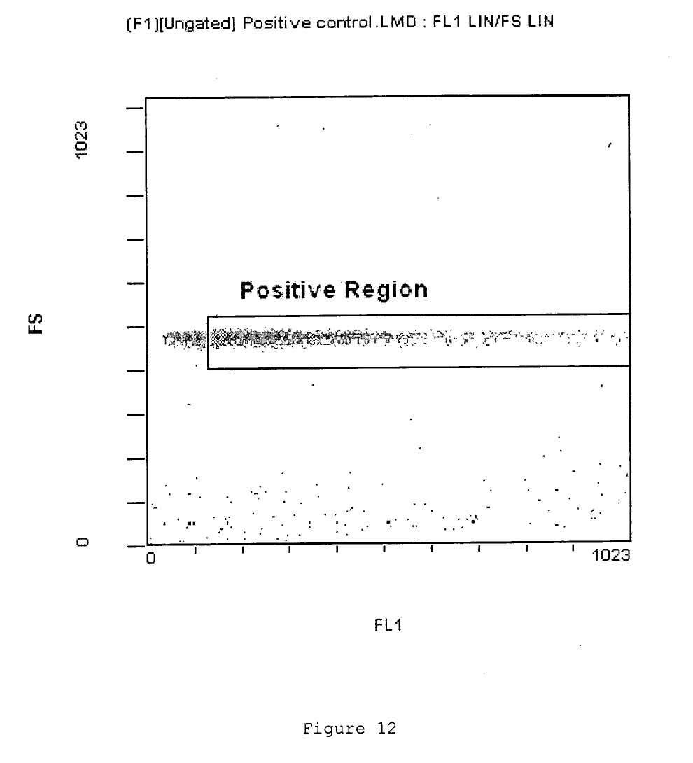

(2) Use Templates as shown in FIGS. 10, 11 and 12.

Note: Results may be interpreted as in the Anti-ENA assay using mean channel shifts in the main population. The dsDNA positive control provided is a cut-off positive sera. Other higher binding products are available.

(3) Positive results may be defined as 1.5 times the mean channel of the negative, normal control.

(4) Transfer of data is customized to either LIS or spreadsheet formats.

The invention is not to be construed as being limited to the specific examples provided herein. Indeed, one important feature of the invention is its flexibility and scope and as well as the fact that it can be utilized in a broad range of circumstances for detecting the presence of a target antigen and other compounds.

1. A no-wash multiplex bead system in which all basic reaction materials are placed into single container via manual application, robotic transfer, or any other batch-type testing procedure and allowed to incubate followed by analysis on a flow cytometer.

2. A system as claimed in claim 1 wherein the container is selected from one or more of test tube(s), micro titer tray, and centrifuge tube.

3. A system as claimed in claim 1 comprising multiple bead sizes, multiple indicator dyes, and test for the presence of multiple constituents.

4. A system as claimed in claim 3 comprising:
(a) selecting from various sizes of beads to be used in the assay;
(b) obtaining antigens or a combination of antigens to be coated on to each bead;
(c) Pre-washing, by centrifugation, a specific amount of beads in a buffer solution;
(d) decanting supernatant from the bead “pellet”;
(e) gently resuspending the bead concentrate;
(f) applying the antigen directly to the bead concentrate;
(g) gently vortexing the bead/antigen solution;
(h) applying another volume of buffer solution;
(i) allowing the bead/antigen solution to incubate;
(j) removing excess antigen and buffer by centrifugation;
(k) carefully decanting supernatant from bead concentrate
(l) gently vortexing the bead/antigen concentrate;
(m) optionally applying a specific amount of protein solution to the bead/antigen concentrate;
(n) gently vortexing bead/antigen solution;
(o) removing excess antigen and protein solution by centrifugation;
(p) decanting the supernatant from the bead/antigen “pellet”;
(q) gently vortexing the bead/antigen solution;
(r) resuspending the bead/antigen mixture to a final assay concentration using buffer;
(s) taking a given amount of the assay mixture and placing into a reaction vessel;
(t) adding a specific amount of the sample to be analyzed into the reaction vessel;
(u) adding the indicator reagent to the same reaction vessel;
(v) gently vortexing and incubating bead/antigen suspension, sample, and indicator solution; and
(w) analyzing on the flow cytometer.

5. A no-wash assay kit for the detection of Anti-ENA antibodies comprising coating beads with RnP/Sm, Sm, SS-A, SS-B, and Scl-70 on sizes 4, 5, 10, 7, and 3 microns, adding indicator antibody to the reaction system along with pre-diluted patient sample, incubating at room temperature, analyzing on the flow cytometer, and generating results based on the outcome of the mean channel fluorescence of the negative, or “normal”, control.

6. A no-wash assay kit as claimed in claim 5 comprising dividing the mean channel into the mean channel of any subsequent samples to determine the index, the value of the index in the determination of positivity being statistically determined by running normal, non-disease patients in significant numbers.

7. A no-wash assay kit as claimed in claim 5 wherein one bead is coated with a specific antigen, incubated with sample and one or multiple fluorescent conjugates and analyzed on the flow cytometer.

8. A no-wash assay kit as claimed in claim 7 wherein the antigen is H. pylori attached to a specific bead, the patient sera is pre-diluted and applied to the coated bead solution along with antibodies to anti-human IgG, IgA, and IgM each with their own unique fluorochrome, and, after incubation, detecting three parameters for each sample.

9. A no-wash assay kit as claimed in claim 5 comprising using a single bead coated with dsDNA, prediluting patient sample and adding to a volume of dsDNA coated beads together with diluted anti-human IgG f(ab′)² FITC, or other fluorochrome, and incubating at room temperature in the dark and reading on a flow cytometer.

10. A no-wash assay kit as claimed in claim 9 wherein the results are provided semi-quantitatively as the mean channel fluorescence of the negative control divided into the mean channel of the sample or the relative positivity of the sample within a selected area pre-established for normalcy.
